(19) United States
(12) Patent Application Publication
(10) Pub. No.: US 2026/0062561 A1
(43) Pub. Date: Mar. 5, 2026

(54) ANTI-MICROBIALLY INDUCED CORROSION BIODEGRADABLE COMPOSITION

(71) Applicant: The United States of America, as represented by the Secretary of the Navy, Crane, IN (US)

(72) Inventor: Armen Kvryan, Glendale, CA (US)

(73) Assignee: The United States of America, as represented by the Secretary of the Navy, Arlington, VA (US)

(21) Appl. No.: 19/312,988

(22) Filed: Aug. 28, 2025

Related U.S. Application Data
(60) Provisional application No. 63/689,337, filed on Aug. 30, 2024.

(51) Int. Cl. C09D 5/08; C08G 63/08; C08K 7/22; C09D 167/04; C12N 1/20; C12R 1/125

(52) U.S. Cl. CPC C09D 5/08; C08G 63/08; C08K 7/22; C09D 167/04; C12N 1/205; C08G 2130/00; C08G 2150/90; C12R 2001/125

(57) ABSTRACT

Provided is an anti-microbially induced corrosion biodegradable composition comprising: a biodegradable polymer base; a degradation modifier; an encapsulated dormant bacteria; an optional encapsulation agent; and an activation trigger. In an illustrative embodiment, the biodegradable polymer base comprises polylactic acid (PLA) or polyhydroxyalkanoates, the degradation modifier comprises Polyethylene glycol (PEG) or Glycerol, the encapsulated dormant bacteria comprises Bacillus subtilis spores, the encapsulation agent comprises calcium alginate or silica microcapsules, and the activation trigger comprises trehalose or glucose. A method of preventing microbially induced corrosion using the disclosed composition is also provided. The inventive composition provides many advantages over other known methods to prevent MIC in that it is significantly cost effective, highly customized, rapidly prototyped, and can be implemented into any material/asset that is already in use.

### CROSS-REFERENCE TO RELATED APPLICATIONS

The present application claims priority to U.S. Provisional Patent Application Ser. No. 63/689,337, filed Aug. 30, 2024, entitled “Additive Manufacturing Composition for Preventing Microbiologically Induced Corrosion,” the disclosure of which is expressly incorporated by reference herein.

### STATEMENT REGARDING FEDERALLY SPONSORED RESEARCH OR DEVELOPMENT

The invention described herein was made in the performance of official duties by employees of the Department of the Navy and may be manufactured, used and licensed by or for the United States Government for any governmental purpose without payment of any royalties thereon. This invention (Navy Case 212236) is assigned to the United States Government and is available for licensing for commercial purposes. Licensing and technical inquiries may be directed to the Technology Transfer Office, Naval Surface Warfare Center Port Hueneme Division, email: Alan.w.jaeger@navy.mil or phone (805) 205-0638.

### FIELD OF THE INVENTION

The field of invention relates generally to corrosion protection. More particularly, it pertains to an anti-microbially induced corrosion biodegradable composition and method of use.

### BACKGROUND

Microbial corrosion, also known as microbially induced corrosion (MIC), or biocorrosion, is a process whereby microbes affect the electrochemical environment of a surface. Microbes build a biofilm on a surface, which can lead to an increase in corrosion of the surface. MIC is a corrosion phenomenon that is not well understood and very difficult to prevent.

Currently, a variety of measures for preventing MIC are known used. These methods, however, provide limited success. One such method involves cathodic protection, which is a method of making a metal surface the cathodic side of an electrochemical cell. This method, however, is cost prohibitive and not feasible in many applications. Another known method involves ultraviolet sterilization, which is designed to kill the microbes. This method, however, provides short term prevention and is not possible for use with submerged assets. Other methods include physical scrapping, which is a labor intensive, short term solution that is not applicable in many cases.

Other methods involve the use of specialized materials that form protective layers or release metal ions that are toxic to the microbes. Theses materials, however are exceedingly expensive. Another material that has been used is a polymer coating over a metallic surface. As can be appreciated, polymer does not provide the same durability and longevity as metal surfaces. A final method of preventing MIC is with biocides. These compositions, however, can be toxic and difficult to contain fallout. As can be appreciated from the above, a new solution to prevent MIC is desirable.

### SUMMARY OF THE INVENTION

The present invention relates to an anti-microbially induced corrosion biodegradable composition comprising: a biodegradable polymer base; a degradation modifier; an encapsulated dormant bacteria; an optional encapsulation agent; and an activation trigger. In an illustrative embodiment, the biodegradable polymer base comprises polylactic acid (PLA) or polyhydroxyalkanoates, the degradation modifier comprises polyethylene glycol (PEG) or glycerol, the encapsulated dormant bacteria comprises Bacillus subtilis spores, the encapsulation agent comprises calcium alginate or silica microcapsules, and the activation trigger comprises trehalose or glucose. A method of preventing microbially induced corrosion using the disclosed composition is also provided.

The composition interacts with the microbes to prevent MIC, and thereby prevent degradation of a surface. The composition essentially eats away/affects the microbes responsible for MIC, and degrades away once the microbes are removed, thereby leaving no remnants. Removal of the microbes in turn, reduces and/or eliminates corrosion induced by the microbes.

The inventive composition provides many advantages over other known methods to prevent MIC in that it is significantly cost effective, highly customized, rapidly prototyped, and can be implemented into any material/asset that is already in use. Furthermore, the biodegradability factor of the composition makes it more environmentally friendly when compared to harsh chemicals that are currently being used.

Additional features and advantages of the present invention will become apparent to those skilled in the art upon consideration of the following detailed description of the illustrative embodiment exemplifying the best mode of carrying out the invention as presently perceived.

### BRIEF DESCRIPTION OF THE DRAWINGS

The detailed description of the drawings particularly refers to the accompanying figures in which:

FIG. 1 shows a view of microbiologically induced corrosion of metallic substrate.

FIG. 2 shows a view of an additive manufacturing composition applied to a surface for preventing microbiologically induced corrosion.

### DETAILED DESCRIPTION OF THE DRAWINGS

The embodiments of the invention described herein are not intended to be exhaustive or to limit the invention to precise forms disclosed. Rather, the embodiments selected for description have been chosen to enable one skilled in the art to practice the invention.

Generally, provided is an anti-microbially induced corrosion biodegradable composition comprising: a biodegradable polymer base; a degradation modifier; an encapsulated dormant bacteria; an optional encapsulation agent; and an activation trigger.

In an illustrative embodiment, the biodegradable polymer base is in the range of 50-80 wt %; the degradation modifier is in the range of 5-15 wt % the encapsulated dormant bacteria is in the range of 1-10 wt % the optional encapsulation agent is in the range of 1-5 wt %; and the activation trigger is in the range of 1-5 wt %. In an illustrative embodiment, the biodegradable polymer base comprises polylactic acid (PLA) or polyhydroxyalkanoates (PHAs). In an illustrative embodiment, the degradation modifier comprises polyethylene glycol (PEG) or Glycerol. In an illustrative embodiment, the encapsulated dormant bacteria comprises Bacillus subtilis spores. In an illustrative embodiment, the encapsulation agent comprises calcium alginate or silica microcapsules. In an illustrative embodiment, the activation trigger comprises trehalose or glucose. In an illustrative embodiment, provided is an anti-microbially induced corrosion biodegradable composition comprising: polylactic acid or polyhydroxyalkanoates; polyethylene glycol or glycerol; encapsulated dormant Bacillus subtilis spores; calcium alginate or silica microcapsules; and trehalose or glucose. In an illustrative embodiment, the polylactic acid or polyhydroxyalkanoates is in the range of 50-80 wt %; the polyethylene glycol or glycerol is in the range of 5-15 wt %; the encapsulated dormant Bacillus subtilis spores are in the range of 1-10 wt %; the calcium alginate or silica microcapsules is in the range of 1-5 wt %; and the Trehalose or Glucose is in the range of 1-5 wt %.

In an illustrative embodiment, provided is an anti-microbially induced corrosion biodegradable composition comprising: polylactic acid or polyhydroxyalkanoates in the range of 50-80 wt %; polyethylene glycol or glycerol in the range of 5-15 wt %; encapsulated dormant Bacillus subtilis spores in the range of 1-10 wt %; calcium alginate or silica microcapsules in the range of 1-5 wt %; and trehalose or glucose in the range of 1-5 wt %.

In an illustrative embodiment, provided is a method of preventing microbially induced corrosion comprising: providing an anti-microbially induced corrosion biodegradable composition comprising: a biodegradable polymer base; a degradation modifier; an encapsulated dormant bacteria; an optional encapsulation agent; and an activation trigger; applying the composition to a surface undergoing corrosion microbially induced corrosion; wherein the composition interacts with one or more microbes causing microbially induced corrosion to prevent degradation of the surface.

In an illustrative embodiment, the biodegradable polymer base is in the range of 50-80 wt %; the degradation modifier is in the range of 5-15 wt % the encapsulated dormant bacteria is in the range of 1-10 wt % the optional encapsulation agent is in the range of 1-5 wt %; and the activation trigger is in the range of 1-5 wt %. In an illustrative embodiment, the biodegradable polymer base comprises polylactic acid or polyhydroxyalkanoates. In an illustrative embodiment, the degradation modifier comprises polyethylene glycol or glycerol. In an illustrative embodiment, the encapsulated dormant bacteria comprises Bacillus subtilis spores. In an illustrative embodiment, the encapsulation agent comprises calcium alginate or silica microcapsules. In an illustrative embodiment, the activation trigger comprises trehalose or glucose.

In an illustrative embodiment, provided is a method of preventing microbially induced corrosion comprising: providing an anti-microbially induced corrosion biodegradable composition comprising: polylactic acid or polyhydroxyalkanoates; polyethylene glycol or glycerol; encapsulated dormant Bacillus subtilis spores; calcium alginate or silica microcapsules; and trehalose or glucose; applying the composition to a surface undergoing corrosion microbially induced corrosion; wherein the composition interacts with one or more microbes causing microbially induced corrosion to prevent degradation of the surface.

In an illustrative embodiment, the biodegradable polymer base is in the range of 50-80 wt %; the degradation modifier is in the range of 5-15 wt % the encapsulated dormant bacteria is in the range of 1-10 wt % the optional encapsulation agent is in the range of 1-5 wt %; and the activation trigger is in the range of 1-5 wt %. In an illustrative embodiment, the biodegradable polymer base comprises polylactic acid or polyhydroxyalkanoates. In an illustrative embodiment, the degradation modifier comprises polyethylene glycol or glycerol. In an illustrative embodiment, the encapsulated dormant bacteria comprises Bacillus subtilis spores. In an illustrative embodiment, the encapsulation agent comprises calcium alginate or silica microcapsules. In an illustrative embodiment, the activation trigger comprises trehalose or glucose.

In an illustrative embodiment, provided is a method of preventing microbially induced corrosion comprising: providing an anti-microbially induced corrosion biodegradable composition comprising: polylactic acid or polyhydroxyalkanoates in the range of 50-80 wt %; polyethylene glycol or glycerol in the range of 5-15 wt %; encapsulated dormant Bacillus subtilis spores in the range of 1-10 wt %; calcium alginate or silica microcapsules in the range of 1-5 wt %; and trehalose or glucose in the range of 1-5 wt %; applying the composition to a surface undergoing corrosion microbially induced corrosion; wherein the composition interacts with one or more microbes causing microbially induced corrosion to prevent degradation of the surface.

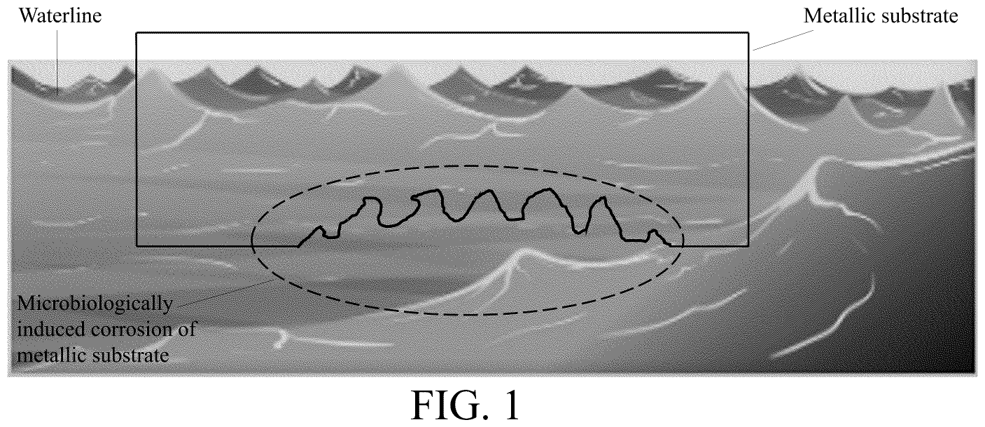

FIG. 1 shows a view of microbiologically induced corrosion of metallic substrate. In an illustrative embodiment, the anti-microbially induced corrosion biodegradable composition comprises a biodegradable polymer base; a degradation modifier; an encapsulated dormant bacteria; an optional encapsulation agent; and an activation trigger.

In an illustrative embodiment, the biodegradable polymer base functions as a matrix for the composition. In an illustrative embodiment, the biodegradable polymer base comprises polylactic acid or polyhydroxyalkanoates in the range of 50-80 wt %. In an illustrative embodiment, the PLA serves as the primary structural matrix. As can be appreciated, PLA is biodegradable and compatible with additive manufacturing (e.g., FDM 3D printing, molding). Furthermore, PLA degrades over time in moist environments to release encapsulated agents. In an illustrative embodiment, PHAs are biodegradable bioplastics produced by microorganism fermentation via a carbon source that function like traditional plastics but are naturally derived and compostable. PHAs can be used as a scaffold/structural matrix in a manner similar to PLAs.

In an illustrative embodiment, the degradation modifier or plasticizer comprises PEG or glycerol in the range of 5-15 wt %. The degradation modifier enhances flexibility, lowers melting temperature, and modulates the degradation rate of PLA.

In an illustrative embodiment, the encapsulated dormant bacteria comprises dormant Bacillus subtilis spores in the range of 1-5 wt %. Bacillus subtilis spores are active in moist, anaerobic conditions and are known to inhibit sulfate-reducing bacteria (SRBs) like Desulfovibrio vulgaris, a major contributor to MIC. Bacillus subtilis spores outcompete and/or neutralize MIC agents via biofilm interference, competition for nutrients, and secretion of antimicrobial compounds.

In an illustrative embodiment, the encapsulation agent comprises calcium alginate or silica microcapsules in the range of 1-5 wt %. The encapsulation agent provides thermal protection and shelf-life stability for the encapsulated dormant bacteria spores during processing and before activation. In an illustrative embodiment, the encapsulation agent is optionally included in the composition.

In an illustrative embodiment, the activation trigger comprises trehalose or another carbon source like glucose in the range of 1-5 wt %. The activation trigger functions as an additive nutrient carrier to supports reactivation and proliferation of the bacteria upon release. In addition, trehalose also stabilizes spores during drying and thermal exposure.

In an illustrative embodiment, the composition comprises: a biodegradable polymer base; a degradation modifier; an encapsulated dormant bacteria; an optional encapsulation agent; and an activation trigger.

In an illustrative embodiment, the composition comprises: polylactic acid or polyhydroxyalkanoates; PEG or glycerol; encapsulated dormant Bacillus subtilis spores; calcium alginate or silica microcapsules; and trehalose or glucose.

In an illustrative embodiment, the composition comprises polylactic acid or polyhydroxyalkanoates in the range of 50-80 wt %; PEG or glycerol in the range of 5-15 wt %; encapsulated dormant Bacillus subtilis spores in the range of 1-10 wt %; calcium alginate or silica microcapsules in the range of 1-5 wt %; and trehalose or glucose in the range of 1-5 wt %.

The components of the composition, wt % and purpose are provided in Table 1.

TABLE 1
components of an anti-MIC biodegradable composition

| Component | wt % | Purpose |
|---|---|---|
| Polylactic acid or | 50-80 | Structural biodegradable matrix |
| polyhydroxyalkanoates | | |
| PEG or Glycerol | 5-15 | Plasticizer and degradation modifier |
| Bacillus subtilis spores | 1-10 | Anti-MIC agent; inhibits SRBs |
| Calcium alginate or silica | 1-5 | Encapsulation/protection of spores |
| Trehalose or glucose | 1-5 | Nutrient/carbon source for bacterial |
| | | growth |
| Inert fillers (e.g., silica) | balance | Mechanical property tuning |

In an illustrative embodiment, the inventive composition can be produced via additive manufacturing. In an illustrative embodiment, the composition can be 3-D printed with several materials layered upon themselves to customize the types of corrosion to attack as a function of time. As can be appreciated, 3-D printing allows for very quick prototyping and provides access to shapes and geometries that would not be possible in other form of manufacturing. Furthermore, 3-D printing allows for material gradation and multimaterial builds. Finally, this method allows for field repairs on site given its small footprint and quick printing capability

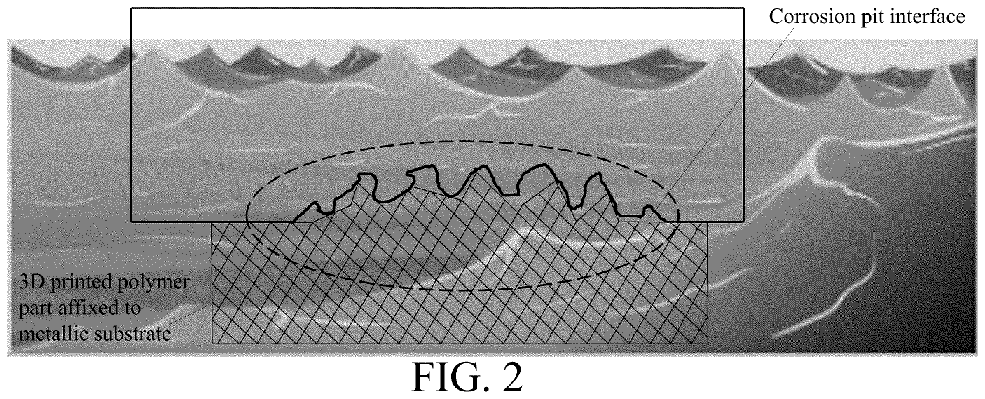

FIG. 2 shows a view of an anti-MIC biodegradable composition applied to a surface for preventing microbiologically induced corrosion. In an illustrative embodiment, the composition is applied to/makes contact with a surface undergoing MIC to slow down and/or eliminate the corrosion occurring by hindering the kinetics of the corrosion. Once the interaction with the microbes responsible for the MIC is complete, the bacteria, along with the rest of the constituents naturally and organically biodegrade into a body of water, thereby reducing/eliminating the MIC issue while leaving no carbon footprint.

In an illustrative embodiment, provided is a method of preventing microbially induced corrosion comprising: providing an anti-MIC biodegradable composition as described above; and applying the composition to a surface undergoing corrosion microbially induced corrosion; wherein the composition interacts with one or more microbes causing microbially induced corrosion to prevent degradation of the surface.

In an illustrative embodiment, the composition comprises: a biodegradable polymer base; a degradation modifier; an encapsulated dormant bacteria; an optional encapsulation agent; and an activation trigger.

In an illustrative embodiment, the composition comprises: polylactic acid or polyhydroxyalkanoates; polyethylene glycol or glycerol; encapsulated dormant Bacillus subtilis spores; calcium alginate or silica microcapsules; and trehalose or glucose.

In an illustrative embodiment, the composition comprises PLA or PHAs in the range of 50-80 wt %; PEG or glycerol in the range of 5-15 wt %; encapsulated dormant Bacillus subtilis spores in the range of 1-10 wt %; calcium alginate or silica microcapsules in the range of 1-5 wt %; and trehalose or glucose in the range of 1-5 wt %.

Alternatively, the inventive composition and method can be used to destroy a surface asset. In an illustrative embodiment, the composition comprises microbes that initiate MIC or another form of corrosion via implementation of oxide particles that lower the surface potential of the surface it is being applied to. As can be appreciated, in this non-limiting embodiment, the inventive composition increases the rate of corrosion and damages the surface in a slow but controlled method.

In an illustrative embodiment, utilizing biodegradable materials that can release certain elements into a surrounding liquid also allows for the altering of the pH of the liquid once it has degraded.

The inventive composition provides many advantages over other known methods to prevent MIC in that it is significantly cost effective, highly customized, rapidly prototyped, and can be implemented into any material/asset that is already in use. Furthermore, the biodegradability factor of the composition makes it more environmentally friendly when compared to harsh chemicals that are currently being used.

Although the invention has been described in detail with reference to certain preferred embodiments, variations and modifications exist within the spirit and scope of the invention as described and defined in the following claims.

1. An anti-microbially induced corrosion biodegradable composition comprising:
a biodegradable polymer base;
a degradation modifier;
an encapsulated dormant bacteria;
an optional encapsulation agent; and
an activation trigger.

2. The anti-microbially induced corrosion biodegradable composition of claim 1, wherein said biodegradable polymer base is in the range of 50-80 wt %; said degradation modifier is in the range of 5-15 wt %; said encapsulated dormant bacteria is in the range of 1-10 wt %; said optional encapsulation agent is in the range of 1-5 wt %; and said activation trigger is in the range of 1-5 wt %.

3. The anti-microbially induced corrosion biodegradable composition of claim 1, wherein said biodegradable polymer base comprises polylactic acid.

4. The anti-microbially induced corrosion biodegradable composition of claim 1, wherein said degradation modifier comprises polyethylene glycol or glycerol.

5. The anti-microbially induced corrosion biodegradable composition of claim 1, wherein said encapsulated dormant bacteria comprises Bacillus subtilis spores.

6. The anti-microbially induced corrosion biodegradable composition of claim 1, wherein said encapsulation agent comprises calcium alginate or silica microcapsules.

7. The anti-microbially induced corrosion biodegradable composition of claim 1, wherein said activation trigger comprises trehalose or glucose.

8. An anti-microbially induced corrosion biodegradable composition comprising:
polylactic acid;
polyethylene glycol or glycerol;
encapsulated dormant Bacillus subtilis spores;
calcium alginate or silica microcapsules; and
trehalose or glucose.

9. The anti-microbially induced corrosion biodegradable composition of claim 1, wherein said polylactic acid is in the range of 50-80 wt %; said polyethylene glycol or glycerol is in the range of 5-15 wt %; said encapsulated dormant Bacillus subtilis spores are in the range of 1-10 wt %; said calcium alginate or silica microcapsules is in the range of 1-5 wt %; and said trehalose or glucose is in the range of 1-5 wt %.

10. An anti-microbially induced corrosion biodegradable composition comprising:
polylactic acid in the range of 50-80 wt %;
polyethylene glycol or glycerol in the range of 5-15 wt %;
encapsulated dormant Bacillus subtilis spores in the range of 1-10 wt %;
calcium alginate or silica microcapsules in the range of 1-5 wt %; and
trehalose or glucose in the range of 1-5 wt %.

11. A method of preventing microbially induced corrosion comprising:
providing an anti-microbially induced corrosion biodegradable composition comprising:
a biodegradable polymer base;
a degradation modifier;
an encapsulated dormant bacteria;
an optional encapsulation agent; and
an activation trigger;
applying said composition to a surface undergoing corrosion microbially induced corrosion;
wherein said composition interacts with one or more microbes causing microbially induced corrosion to prevent degradation of said surface.

12. The method of claim 11, wherein said biodegradable polymer base is in the range of 50-80 wt %; said degradation modifier is in the range of 5-15 wt %; said encapsulated dormant bacteria is in the range of 1-10 wt %; said optional encapsulation agent is in the range of 1-5 wt %; and said activation trigger is in the range of 1-5 wt %.

13. The method of claim 11, wherein said biodegradable polymer base comprises polylactic acid.

14. The method of claim 11, wherein said degradation modifier comprises polyethylene glycol or glycerol.

15. The method of claim 11, wherein said encapsulated dormant bacteria comprises Bacillus subtilis spores.

16. The method of claim 11, wherein said encapsulation agent comprises calcium alginate or silica microcapsules.

17. The method of claim 11, wherein said activation trigger comprises trehalose or glucose.

18. A method of preventing microbially induced corrosion comprising:
providing an anti-microbially induced corrosion biodegradable composition comprising:
polylactic acid;
polyethylene glycol or glycerol;
encapsulated dormant Bacillus subtilis spores;
calcium alginate or silica microcapsules; and
trehalose or glucose;
applying said composition to a surface undergoing corrosion microbially induced corrosion;
wherein said composition interacts with one or more microbes causing microbially induced corrosion to prevent degradation of said surface.

19. The method of claim 18, wherein said biodegradable polymer base is in the range of 50-80 wt %; said degradation modifier is in the range of 5-15 wt %; said encapsulated dormant bacteria is in the range of 1-10 wt %; said optional encapsulation agent is in the range of 1-5 wt %; and said activation trigger is in the range of 1-5 wt %.

20. The method of claim 18, wherein said biodegradable polymer base comprises Polylactic Acid.

21. The method of claim 18, wherein said degradation modifier comprises polyethylene glycol or glycerol.

22. The method of claim 18, wherein said encapsulated dormant bacteria comprises Bacillus subtilis spores.

23. The method of claim 18, wherein said encapsulation agent comprises calcium alginate or silica microcapsules.

24. The method of claim 18, wherein said activation trigger comprises trehalose or glucose.

25. A method of preventing microbially induced corrosion comprising:
providing an anti-microbially induced corrosion biodegradable composition comprising:
polylactic acid in the range of 50-80 wt %;
polyethylene glycol or glycerol in the range of 5-15 wt %;
encapsulated dormant Bacillus subtilis spores in the range of 1-10 wt %;
calcium alginate or silica microcapsules in the range of 1-5 wt %; and
trehalose or glucose in the range of 1-5 wt %;
applying said composition to a surface undergoing corrosion microbially induced corrosion;
wherein said composition interacts with one or more microbes causing microbially induced corrosion to prevent degradation of said surface.
